(19) United States
(12) Patent Application Publication
(10) Pub. No.: US 2026/0224222 A1
(43) Pub. Date: Aug. 6, 2026

(54) IMPLANT DELIVERY SYSTEM WITH ELECTROLYTIC DETACHMENT

(71) Applicant: KFNT HOLDINGS, LLC, Germantown, TN (US)

(72) Inventors: Stephen J. Hebert, San Francisco, CA (US); Bartosz Bojanowski, Redwood, CA (US); Ty Matthew Farkas, Mountain View, CA (US)

(73) Assignee: KFNT HOLDINGS, LLC, Germantown, TN (US)

(21) Appl. No.: 19/155,041

(22) PCT Filed: Jan. 26, 2024
(86) PCT No.: PCT/US2024/013051

§ 371 Date: Aug. 8, 2025

Related U.S. Application Data
(60) Provisional application No. 63/444,000, filed on Feb. 8, 2023.

(51) Int. Cl. A61B 17/12

(52) U.S. Cl. CPC A61B 17/1214; A61B 17/12113; A61B 2017/12063

(57) ABSTRACT

A system for delivering a vascular implant into a body lumen of a patient having an elongated delivery device having an outer member and a delivery member positioned within the lumen of the outer member. The delivery member has an engagement member at a distal portion and a detachment zone spaced proximally of the engagement member. The engagement member is engageable with a first engagement portion of the implant. Energy is applied to the delivery member to detach the engagement member from the delivery wire and separate the implant along with the engagement member from the delivery member.

### BACKGROUND

This application claims priority from provisional application 63/444,000, filed Feb. 8, 2023, the entire contents of which are incorporated by reference herein.

### TECHNICAL FIELD

This application relates to delivery systems for surgical implants, and more particularly, to delivery systems with electrolytic detachments.

### BACKGROUND OF RELATED ART

An aneurysm is a localized, blood-filled balloon-like bulge that can occur in the wall of any blood vessel, as well as within the heart. There are various treatments for aneurysms. One endovascular treatment option for aneurysms is complete reconstruction of the damaged vessel using a stent-graft or other vascular prothesis. While stent-grafts are useful in treating aneurysms in other parts of the body, they are not a treatment option for intracranial aneurysms due to the risk of cutting off blood flow to feeder vessels that may be vital for brain function. Stent-grafts can also be stiff, hard to deliver/retract, and can be highly thrombogenic within the parent vessel, all of which are undesirable features for intracranial aneurysm treatment. The current standard vascular prosthesis for treating brain aneurysms is the flow diverter. Flow diverters are metallic open cell braids that are deployed in the parent vessel to cover the aneurysm in order to divert or at least reduce flow into the aneurysm sac. While these devices are successful at slowing flow, they pose thrombo-embolic risk and require patients to be on antiplatelet drugs and sometimes result in the aneurysm rupturing over time.

More recently, self-expanding mechanical endosaccular devices have been introduced to treat brain aneurysms. These devices are designed to either expand at the base of the aneurysm to block the aneurysm neck or within the sac of the aneurysm. The goal of both designs is to reduce the flow of blood into the aneurysm with the purpose of obliteration of the aneurysm. While these devices have proven effective, there is still a percentage of cases requiring retreatment. Due to endosaccular device designs, retreatment options are limited and usually require the use of microcoils.

Prior to the introduction of flow diverters and self-expanding endosaccular devices, treatment of intracranial aneurysms centered on packing or filling an aneurysm with microcoils made primarily of platinum. The goal of coiling is to achieve a sufficient packing density to eliminate circulation of blood, which leads to thrombus formation and aneurysm closure over time. The major benefit of this method is that it does not require the use of antiplatelet drugs. However, if the aneurysm is underpacked, compaction may occur, which requires retreatment.

Various modifications to microcoils have been attempted over the years in an effort to overcome compaction. Early modifications to platinum coils included the addition of thrombogenic materials. One of the first thrombogenic coil designs had thrombogenic fibers secured radially along its length. This resulted in a bulky coil that could not be easily delivered through newly introduced 0.0165″ ID microcatheters. The design also allows clot to build up between fibers resulting in possible emboli that could dislodge and result in a stroke.

Another design incorporated an open cell braid made of polymer that was placed over the coil. While this design streamlined the profile of the coil for delivery, the windings of the coil were still left exposed allowing flow through the coil and not creating sufficient blood stagnation.

Later designs incorporated polymer coatings that covered the entire length of the coil. One design employed a polymer coating that was bioabsorbable. While this design ensured that the coil had a solid wall to help stop flow, the coil was too stiff and potentially absorbed too fast resulting in pockets that led to compaction over time. Another design incorporated a hydrogel that undergoes a progressive expansion to increase packing density after initial filling. While this design did succeed in slowing flow with both a solid wall and a higher potential packing density, early cases had a high incidence of delayed hydrocephalus related to this coil type. At the time, the mechanism by which hydrogel coils may induce hydrocephalus remained poorly understood.

While the above devices attempted to treat intracranial aneurysms with minimally invasive techniques, there was still a need for a highly compliant and thrombogenic filler that blocks blood flow within the sac of the aneurysm without the foregoing drawbacks. The micrografts of commonly assigned U.S. Pat. Nos. 9,999,413, 10,736,730, 10,857,012, and 10,925,611 met this need. These patents disclose devices that advantageously achieve sufficient flexibility to enable advancement through the tortuous vasculature into the cerebral vasculature and high packing densities while maintaining a high concentration of thrombogenic material. These devices also cause rapid clotting of the blood and promote tissue ingrowth within a relatively short period of time. The devices are soft, compressible and absorbent to retain blood. These devices are designed for minimally invasive insertion, and are easy to deliver and deploy at the intracranial site as well as manufacturable in a small enough size for use in cerebral vasculature. These devices are constructed to effectively pack the aneurysm without damaging the sac or other tissue while promoting rapid clotting and healing of an intracranial aneurysm with reduction in mass effect.

Delivery of the implants of these patents as well as other types of implants described above can be challenging, especially with implants having a secondary shape that is helical or three-dimensional i.e., complex shapes.

The aforedescribed vascular implants are delivered through the vasculature into the aneurysm. Some devices have a mechanical attachment in which the delivery member mechanically secures the implant and then releases the implant at the surgical site. Other devices utilize energy, such an electrical or thermal energy to detach the implant. In many of these devices, the implant is electrolytically detached from the delivery member.

The need exists to improve delivery and implantation of such vascular implants, especially to improve current electrolytic detachments.

### SUMMARY OF INVENTION

The inventors of U.S. Pat. Nos. 9,999,413, 10,736,730, 10,857,012 and 10,925,611 developed an improved electrolytic detachment of the implant from the delivery member utilizing a bipolar system design. The improved delivery systems of the present invention include an outer conductive member and an inner wire with a tip, preferably a J-shaped tip or an enlarged or bulbous tip, e.g., with a ball end, which engages the implant. Application of electrical energy through the wire causes the tip of the wire to detach from the delivery wire, thereby detaching the implant. The anode and cathode of the delivery system are preferably close together or near the detachment zone. Further details of the delivery system of the present invention are discussed in detail below.

In accordance with one aspect of the present invention, a system for delivering a vascular implant into a body lumen of a patient is provided comprising an elongated delivery device having an outer member having a lumen and a delivery member positioned within the lumen of the outer member. The delivery member has an engagement member at a distal portion and a detachment zone spaced proximally of the engagement member. The engagement member is engageable with a first engagement portion of the implant and the proximal region of the delivery member extends proximally of the outer member, wherein energy is applied to the delivery member to detach the engagement member from the delivery member and separate the implant along with the engagement member from the delivery member.

In some embodiments, bipolar energy is applied to detach the delivery member from the implant wherein the delivery member forms the anode and the outer member forms the cathode.

In some embodiments, the engagement member has a J-tip engageable with the implant. In some embodiments, the implant includes a tube at a proximal end having a recess, and the recess forms the first engagement portion dimensioned to receive the J-tip. In other embodiments, the engagement member has an enlarged end such as a ball tip and the recess forms the first engagement portion dimensioned to receive the ball end.

In some embodiments, the outer member includes an hypotube forming a conductive member, and the delivery member extends distally therefrom. In some embodiments, a proximal region of the delivery member is offset from a central longitudinal axis of the conductive member.

The system can further comprise in some embodiments a conductive member positioned radially of the delivery member, the delivery member and conductive member in electrical contact and forming an anode of a circuit when the conductive member is electrically coupled to a power supply.

In some embodiments, a flexible wire at a proximal region of the delivery device extends proximally of the outer member to enable bending of the proximal region without kinking. In some embodiments, the flexible wire is positioned radially of the delivery member and distally of the conductive member, and the conductive member terminates proximally of the outer member.

The system can include a first insulator (insulating member) positioned proximal of the detachment zone and a second insulator (insulating member) positioned on the engagement member to insulate the engagement member and prevent electrical energy flow to the implant.

In some embodiments, the proximal region of the delivery device is more flexible than an intermediate and/or distal region of the delivery device.

In some embodiments, the system further comprises a ring assembly at a distal region of the delivery member, the delivery member extending within the ring assembly. In other embodiments, the delivery member is inside a coil at the distal region, and the coil and outer member form the cathode.

In some embodiments, the delivery member at a proximal end loops to form a U-shape. In some embodiments, a coil is provided at a proximal end of the delivery system, and the U-shaped end of the delivery member is in electrical contact with the coil.

In accordance with another aspect of the present invention, a system for delivering a vascular implant into a body lumen of a patient is provided comprising an outer member and an elongated delivery member having an engagement member at a distal portion and a detachment zone spaced proximally of the engagement member, the engagement member engageable with a first engagement portion of the implant. The elongated delivery member is positioned within the outer member and extends distally and proximally of the outer member. A conductive member is positioned proximally of the outer member and in contact with the delivery member for connection to a power source, wherein energy is applied to the conductive member and to the delivery member to detach the engagement member from the delivery member and separate the implant from the delivery member.

In some embodiments, the conductive member is external of the delivery member.

In some embodiments, the system is a bipolar system and the delivery member forms the anode and the outer member forms the cathode.

In some embodiments, the delivery member has a looped end to provide multiple contact areas with the conductive member. In some embodiments, the conductive member is a coil. In some embodiments, the coil has a larger diameter than the diameter of the outer member.

The system in some embodiments has a wire extending distally of the conductive member and a distal end of the conductive member terminates proximally of the outer member and distally of a proximal end of the delivery member. In some embodiments, the wire has a first portion within the outer member and a second portion proximal of the outer member.

In some embodiments, the delivery member at a proximal end loops to form a U-shape. In some embodiments, a coil is provided at a proximal end of the delivery system, and the U-shaped end of the delivery member is in electrical contact with the coil.

In accordance with another aspect of the present invention, a system for delivering a vascular implant into a body lumen of a patient is provided comprising an elongated delivery device having an outer member having a lumen and a delivery member positioned within the lumen of the outer member. The delivery member has an engagement member at a distal portion and a detachment zone spaced proximally of the engagement member. The engagement member is engageable with a first engagement portion of the implant. The delivery member has a ring assembly positioned around a distal portion thereof and has a first portion within the outer member and a second portion extending distally of the outer member, wherein energy is applied to the delivery member to detach the engagement member from the delivery wire and separate the implant along with the engagement member from the delivery member.

In some embodiments, the ring assembly includes a ring having an arm extending proximally within the outer member.

In some embodiments, the detachment zone is distal of the ring assembly.

In some embodiments, the delivery member has a looped proximal end.

### BRIEF DESCRIPTION OF THE DRAWINGS

So that those having ordinary skill in the art to which the subject invention appertains will more readily understand how to make and use the surgical apparatus disclosed herein, preferred embodiments thereof will be described in detail hereinbelow with reference to the drawings, wherein:

FIG. 1 is a cross-sectional view of one embodiment of the bipolar delivery system of the present invention;

FIG. 2A is an enlarged view of the area of detail A of FIG. 1;

FIG. 2B is an enlarged view of the area of detail B of FIG. 1;

FIG. 3 is a side view showing a conventional delivery sheath having a bend;

FIGS. 4A, 4B, 4C and 4D are side views showing the delivery member as it progresses through the bent region of the conventional sheath of FIG. 3 in a delivery device lacking the flexible proximal region of the present invention;

FIG. 5 is a side view showing the delivery wire of the delivery device advanced past the end and exiting the proximal end of the conventional delivery sheath of FIG. 3;

FIGS. 6A, 6B, 6C and 6D are side views showing the delivery device of the present invention having a flexible proximal region as it progresses through the bent region of the conventional sheath of FIG. 3;

FIG. 7A is a side view of the delivery device of FIG. 1 engaged (attached) with the implant;

FIG. 7B is a cross-sectional view showing engagement of the J-tip of the delivery device of FIG. 7A engaged with the vascular implant;

FIG. 8 is a cross-sectional view of an alternate embodiment of the delivery device of the present invention engaged with the implant;

FIG. 9 is a cross-sectional view of an alternate embodiment of the delivery device of the present invention;

FIG. 10 is a cross-sectional view of an alternate embodiment of the delivery device of the present invention showing the proximal region; and

FIG. 11 is a cross-sectional view of an alternate embodiment of the delivery device of the present invention showing the proximal region and FIG. 12 is a cross-sectional view showing the proximal and distal regions of the device of FIG. 11.

### DETAILED DESCRIPTION

The present invention provides delivery systems for micrografts (implants) described in detail in commonly assigned U.S. Pat. No. 10,925,611 (hereinafter the '611 patent), and U.S. Pat. No. 10,857,012 (hereinafter the '012 patent) and U.S. Pat. No. 10,736,730. The entire contents of each of these applications are incorporated herein by reference. Initially, a brief discussion of the micrograft is provided, followed by a discussion of the delivery system of the present invention. Note that further details of the implant, and alternate embodiments thereof, are discussed in detail in the '611 patent and the '012 patent.

It should be appreciated that the delivery systems of the present invention can be used for implants other than those described in the '611 and '012 patent.

FIGS. 4F-4M of the '012 patent show views of one embodiment of an intra-aneurysmal micrograft for insertion into an intracranial aneurysm. The micrograft has a biocompatible non-self-expandable absorbent braided polymeric textile tubular body that is crimped to reduce stiffness and increase wall thickness and fabric density. The micrograft has sufficient stiffness as well as sufficient flexibility. It further is structured to enable a triple capillary action to promote blood clotting. The micrograft further preferably has a high surface area for increased blood absorption, is radially deformable, has a low friction surface for ease of delivery and can be shape set to enhance packing of the aneurysm. The micrograft is especially designed to induce blood stagnation or clot to rapidly treat the aneurysm. The micrograft is configured for delivery to an intracranial aneurysm, although it can be utilized for occlusion in aneurysms in other areas of the body as well as for occlusion in other vascular regions or in non-vascular regions.

The micrograft is constructed of multi-filament interlaced yarns, wherein each yarn is composed of a plurality of polyester filaments having pores or spaces therebetween, and the plurality of yarns also have pores or spaces therebetween. Blood flows through the micrograft in a distal to proximal direction. The micrograft can be navigated to and into the cranial vasculature for placement within a cranial vessel.

Each of the multi-filament yarns are made of multiple wettable micro-filaments, or fibers, assembled with spaces (pores) between them. The pores are sufficiently sized to induce capillary action when contacted by a liquid, resulting in the spontaneous flow of the liquid along the porous yarn (i.e., wicking). This capillarity action between fibers (intra-fiber) within the yarn is termed as “micro-capillary” action. As a result, a sufficiently wettable and porous yarn has high wickability and transports liquid along its length. The multiple filaments also provide a high surface area and can be hydrophilic or hydrophobic.

This assembly of the two or more wickable multi-filament yarns into a permeable structure (such as a textile) results in a “macro-capillary” action, i.e., the transporting of liquid between the yarns and throughout the structure.

The multi-filament yarns can be assembled into a textile tubular structure using a braider or other textile manufacturing equipment and methods.

The vascular graft (micrograft) has a proximal opening at the proximal end and a distal opening at the distal end for blood flow into the distal end and through the lumen (the proximal and distal openings aligned with a longitudinal axis), thereby forming a conduit for transport of blood through the continuous inside lumen (inside diameter). A capillary effect is created within the vascular graft when the biocompatible structure is exposed to blood such that blood is transported in a proximal direction through the distal opening in the vascular graft and through the vascular graft wherein blood clots. Thus, blood initially flows through the distal opening, through the vascular graft and towards the proximal end of opening, with blood quickly stagnating within the graft. In some instances, blood will exit the proximal opening (e.g., if there is sufficient pressure); in other instances, capillary action will only fill the graft and not cause flow out the proximal opening. In other embodiments, the proximal end does not have a proximal opening. The vascular graft retains blood, and becomes saturated with blood, to promote clotting. The outer member, i.e., the textile structure, is configured as a tubular member for flow therein, functioning as a capillary tube. The tubular textile member is configured in a closed cell fashion so as to form a tube for flow therethrough, i.e., the lumen inside the textile structure is sufficiently small to enable function as a capillary tube, but the textile structure still has sufficient sized openings/spaces for absorbing blood through and along the yarns and filaments as described herein. Thus, a continuous wall (continuous inner diameter) is formed along the length of the textile structure to retain blood while also maintaining small spaces (micro-capillaries) in between fibers to wick and absorb blood. This non-expanding closed cell or tight textile, e.g., braided, structure is maintained since the diameter of the textile structure (and thus the diameter of the vascular graft) does not change from the delivery to implant positions.

The tubular textile structure (which forms a braid in some embodiments) forms a continuous circumferential wall along a length without large spaces between the filaments and/or yarns. This continuous wall is shown in the tight spacing of FIGS. 4A-4D of the '611 patent and thus creates a continuous outer member (low porosity wall) to contain and direct flow. The yarns of the textile structure are close enough to form a continuous wall to wick and transfer blood via the wall and inside lumen.

The capillary spaces formed between yarns are termed macro-capillary and capillary spaces formed between individual fibers of a yarn are termed micro-capillary. The capillary action occurs as the yarns making up the wall of the textile structure and the fibers making up the yarns are assembled close enough, as shown in FIGS. 4A and 4M of the '611 patent, to create micro-capillaries that induce wicking. Thus, the tubular textile structure utilizes the three capillary actions (i.e., inside (inner) lumen, inter-yarn and inter-filament capillary actions) to act as a capillary tube and also achieves blood retention inside the tubular structure.

By forming the textile structure as a tubular member (rather than winding/ braiding the filaments about an inner element), and then inserting/positioning the inner core element therein for attachment to the outer textile structure, portions of the inner surface of the inner wall of the textile structure are in contact with the inner element.

The micrograft includes a permanent core element formed of a metal coil having a lumen therein.

Due to the manufactured tube's relatively small inner diameter and a sufficiently dense interlacing braid pattern (i.e., a filamentary wall structure with sufficiently small pore size such that it retains fluid), the third capillary effect is created. When properly sized, this third capillary effect is responsible for spontaneous flow of liquid inside the micrograft lumen, e.g., within the lumen of the braid, in a proximal direction.

To reduce stiffness to assist delivery and packing of the aneurysmal sac, the micrograft tubular body (braid) is crimped during manufacture, i.e., longitudinally compressed and heat set. As the braid is compressed, axial orientation of the braided strands is reduced thereby increasing braid angle with respect to the longitudinal axis of the tubular body which reduces their influence on overall stiffness of the structure, much like a straight wire taking on a more flexible form when coiled. Crimping also effectively increases the picks per inch (PPI), wall thickness, and linear density of the braid by axially compressing the structure and filament bundles. This compression causes an outward radial expansion and an increase in wall thickness of the tube. The resulting braid is much more deflectable, has reduced bend radius, a higher density and up to 2× to 3× or higher increase in PPI, depending on braid structure and compression of length during manufacturing.

This axial compression also causes the braid structure to “snake” or produce a spiral wavy forming a series of macro peaks and valleys, termed “macro-crimps”, in a sinusoidal shape.

FIGS. 4A, 4B, 4D and 4E-4M of the '611 patent show an embodiment of the micrograft having a core element having a lumen for blood flow in the aforementioned capillary effect. The core element is a coil formed into a helical shape and the lumen extends through the coil from the proximal end to the distal end. The coil can be composed of a metal such as platinum or a platinum tungsten alloy. In manufacture, the textile structure in the form a tubular braid is positioned over the coil. The braid is formed separately into a tubular shape with a lumen or longitudinally extending opening extending from the proximal end to the distal end for receipt of the coil. The braid is preferably composed of PET or other thrombogenic material and is preferably substantially a closed cell design to provide a large percentage of outer surface area for contact with the blood and/or vessel/aneurysm wall, but has spaces between the yarns and filaments to enable blood flow into and/or through the device to achieve the capillary effects. The micrograft, with the braid and attached inner coil, is formed into a helical coil shape as shown in FIG. 4K with a lumen extending along its length.

This configuration of the embodiment of FIG. 4A of the '611 patent also encourages rapid blood clotting and, in some instances, clotting can occur immediately upon implantation. When the micrograft (implant) is held in the delivery system within the vessel/aneurysm but prior to release from the delivery system, the micrograft becomes filled partially or entirely with blood so that blood stagnation can commence even before the micrograft is released and implanted, thereby expediting thrombus formation. Saturation of the micrograft in the delivery assembly and once implanted accelerates and/or improves thrombosis.

Note the braid fibers are not only thrombogenic (attract blood platelets and proteins which promote clot) due to their material, e.g., PET can be used as the filaments or as a thrombogenic surface, but also promote stasis as the braid structure traps blood.

A tube, preferably composed of Nitinol, is seated within proximal coils of the helical core element (coil), e.g., screwed or twisted into the proximal coil windings of the helical core element to provide structure for engagement with a delivery device. The braid is melted onto the tube, and the tube extends proximally of the core element. It also extends proximally of the tubular textile structure so a proximal region is exposed for engagement by a delivery member. A distal portion of the tube is within the tubular textile structure.

As noted above, the braid of the implant is preferably non-expandable. That is, after formed, a dimension measured through a transverse cross-section of the implant (braid and coil) is the same in a delivery position within a delivery member as in the placement position. The implant, however, may be stretched to a reduced profile position for delivery and then released for placement to assume its coil shape discussed above. However, when it moves from the delivery to the placement position, the braid does not expand. The change is to the implant (braid and coil) from the linear shape within the delivery member to its secondary shape (helical, framer, etc.) within the body, but the combined thickness of the braid and coil (i.e., the outer diameter of the braid) remains constant during delivery and placement. This is in contrast to expandable braids wherein the diameter of the braid increases when exposed from the delivery member and in the placement position.

The implant of the '611 patent, as described, can be shape set into any complex three-dimensional configuration such as a cloverleaf, a figure-8, a flower-shape, a vortex-shape, an ovoid, randomly shaped, substantially spherical shape, etc. The soft metal coil within the braid aids in visualization. If stiffness of such metal coil is sufficiently low, the secondary shape-set of the polymer braid will drive the overall shape of the device. In other words, the secondary shape of the braid molds the unshaped metal coil which normally shape sets at temperatures much greater than the glass transition temperature of polymers.

The implant is preset to a non-linear configuration and advanced to the aneurysm in a substantially linear configuration and then returns to the same non-linear configuration or different non-linear configuration when delivered into the aneurysm, depending on the space within the aneurysm.

As the micrograft is deployed into the aneurysm, it will take on any preset secondary shapes and random shapes due to contact with the aneurysm walls.

As discussed in the '611 patent, the delivery wire for the implant can be a guidewire. Therefore, if desired, the micrograft delivery system with guidewire can be loaded into the microcatheter prior to catheter placement. The entire assembly, microcatheter and micrograft delivery system, can then be tracked to the aneurysm site using the delivery system's guidewire as the primary tracking wire. The micrograft can alternatively be constructed to mate with other microcoil delivery systems that provide a timed and controlled release, e.g., electrolytic detachment. Such electrolytic detachment systems are discussed in detail below.

The vascular implant can be packaged as described in the '611 patent and in U.S. Patent Publication 20230233279, the entire contents of both applications incorporated herein by reference.

The packaging includes a container (also referred to herein as the implant holder), a long packaging hoop and a short packaging hoop. The hoops are in the form of tubes composed of a material such as HDPE or polypropylene that can be wrapped as shown without significant kinking that could inhibit movement of the components within the tubes. Contained within the tubes are a delivery member and a delivery sheath. The implant is held within the packaging in its secondary shape (its placement state/ condition), which in some embodiments is helical. For delivery to the patient, the implant is pulled into the delivery sheath to assume a more straightened configuration.

Various attachments for the delivery member and implant can be utilized so the implant can be pulled by the delivery member from the container and into the delivery sheath.

The delivery systems of the present invention will now be discussed. The delivery systems of the present invention provide bipolar detachment of the implant (micrograft) once positioned in the aneurysm.

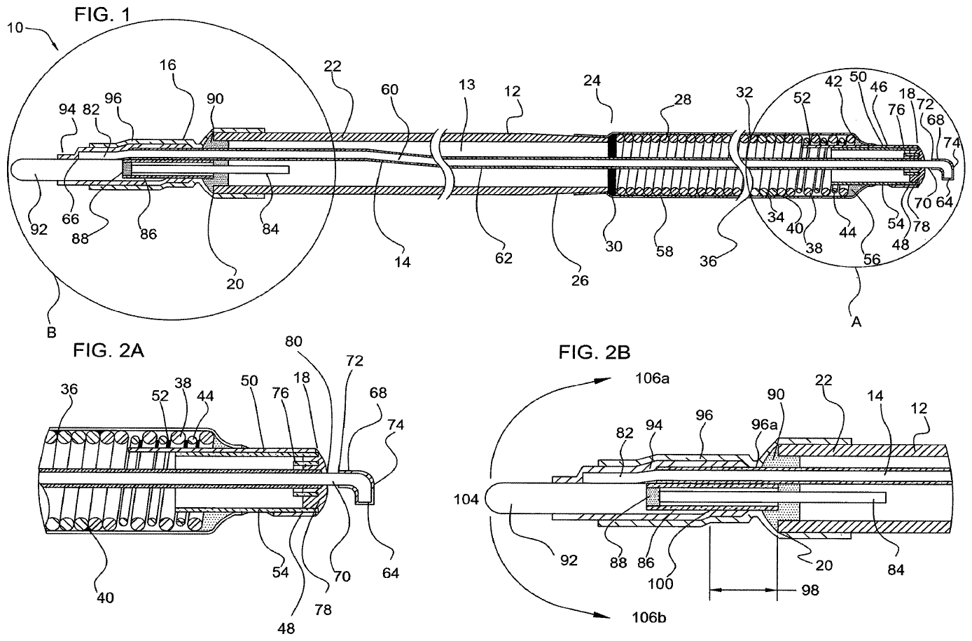

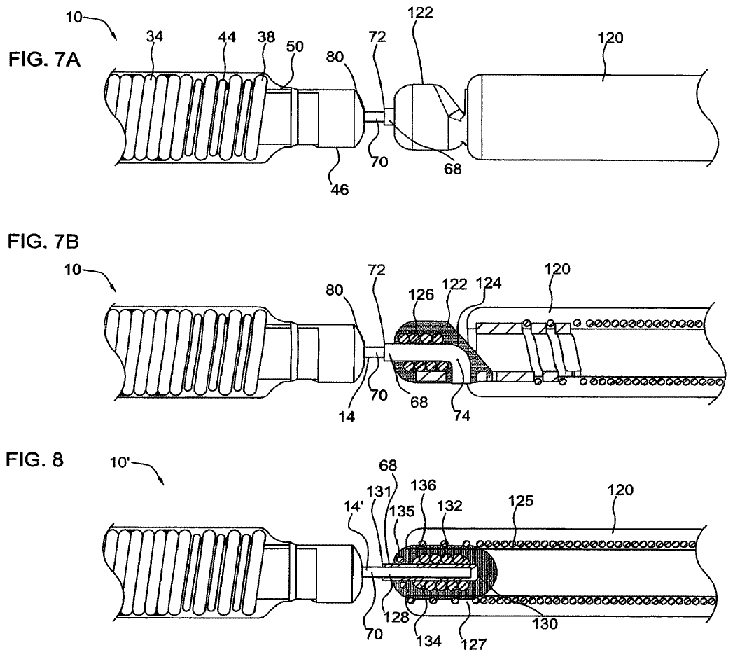

With reference now to the drawings wherein like reference numerals identify similar structural features of the devices disclosed herein throughout the several views, the delivery device 10, as shown in FIGS. 1, 2A and 2B in accordance with one embodiment, has an outer shaft 12 (also referred to herein as an outer member or sheath) and an inner delivery core wire 14 (also referred to herein as a delivery member or inner member) positioned within a lumen of the outer shaft 12 and which is attached at its distal end to the implant (micrograft). The implant has a proximal tube which has an opening to receive the J-tip of the delivery wire 14 as shown in FIGS. 7A and 7B discussed below, although other shaped tips of the delivery wire, e.g., ball tip, are also contemplated.

As shown in FIG. 1, the delivery wire 14 extends through the lumen 13 of the outer shaft 12 and extends distally and proximally of the respective distal and proximal ends 18, 20 of shaft 12. As used herein, the distal end refers to the end closer to the implant, (e.g., the distal end is attached to the implant) and the proximal end is at the opposing end, further from the implant. The delivery device 10 has a flexible proximal end 16. In some embodiments, the flexible end 16 has the ability to bend up to 90 degrees and recover to its original position. The outer shaft 12 includes a hypotube 22 and coils 28, 34, 38 and 44. The hypotube 22 can have a length of about 130 cm to about 160 cm, and preferably about 152 cm, and can have an OD (outer diameter) of about 0.010″ (inches) to about 0.035″, and more preferably about 0.013″, and can have an ID (inner diameter) in the range of about 0.005″ to about 0.020″, and more preferably of about 0.007″. The hypotube 22 can be constructed of material such as stainless steel, nitinol or other conductive material. Hypotube 22 extends from proximal end 20 to distal end 24. At the distal region, the hypotube 22 has a taper 26 which can have a length between about 8 cm and about 15 cm, and preferably about 11 cm, tapering in a distal direction. As noted herein, these dimensions (and other dimensions disclosed herein) are provided by way of example as other dimensions are also contemplated.

Hypotube 22 is laser welded at a distal end to coil 28 with weld 30. Coil 28 is preferably a closed pitch stainless steel coil wound using a stainless-steel wire having a diameter between about 0.001″ and about 0.004″, and preferably about 0.0025″. Coil 28 is generally about 35 cm to about 45 cm in length, and preferably about 37 cm in length, and extends distally from weld 30 to distal end 32. This coil 28, as well as other coils in the assembly, may be open or closed pitch and constructed of material other than stainless steel. Coil 28 preferably has an OD/ID similar to the non-tapered region of hypotube 22 with a preferred ID about 0.007″ and a preferred OD about 0.012″. At distal end 32, coil 28 is welded to radiopaque coil 34 with weld joint 36. Radiopaque coil 34 is wound using between about 0.001″ and about 0.004″, and preferably about 0.0025″ OD, platinum/tungsten wire and has a length of about 1.5 mm to about 5 mm, and preferably about 3 mm and an OD/ID preferably similar to coil 28. In use, coil 34 is lined up under fluoroscopy with a matching marker band on a microcatheter to assist in coil detachment. Radiopaque coil 34 is welded to coil 38 at weld joint 40. Coil 38 is a stainless-steel coil which runs from weld joint 40 to distal end 42 with an OD preferably similar to coil 34. Coil 38 is wound with a stainless-steel wire having a diameter in the range of about 0.0001″ to about 0.004″, and preferably about 0.0025″, and generally has a length of about 1 cm to about 3 cm, and preferably about 2.2 cm. The coil 34 can be open or closed pitch. Intertwined at the distal region is a smaller platinum/tungsten coil 44 that is wound or screwed into coil 38. Platinum coil 44 is wound using platinum/tungsten wire having a diameter between about 0.005 and about 0.0025 inches, and preferably about 0.009″ and can run from about 2 mm to about 5 mm in length with an OD less than that of coil 38. Platinum coil 44 is used to aid in attaching the ring assembly 46. It is not intended for radiopacity although in some embodiments it could be radiopaque. As shown, coil 34 is distal of coil 28, and coil 38 and coil 44 are distal of coil 34.

It should be appreciated that the above-discussed materials and dimensions for the coils are provided by way of example as coils of other dimensions (OD, ID, length, etc.) and other materials are also contemplated.

At distal end 18 of the delivery device is a nitinol ring assembly 46. Ring assembly 46 is constructed from a nitinol tube that in some embodiments has an OD of between about 0.008″ and about 0.016″, and more preferably about 0.010″, and an ID of between about 0.006″ and about 0.0011″, and more preferably about 0.007″. The ring assembly 46 generally has a length between about 0.2 cm and about 3 cm, and preferably about 0.7cm, but can be lengthened or shortened to assist in tip flexibility. Other dimensions are also contemplated as well as other materials such as stainless steel. The nitinol tube is laser cut so that it forms a distal ring 48 with an arm extension 50 that extends proximally so that it enters the distal end of coil 38 and extends to a proximal region of coil 38 where it is welded to coil 38 and platinum coil 44 at weld joint 52. In the illustrated embodiment, arm 50 terminates at a proximal end adjacent the distal end of coil 34. The preferred length for the combined assembly of coil 34, coil 38 and ring assembly 46 is about 3.2 cm (32 mm) from the proximal end of coil 34 (weld joint 36) to the distal end of ring assembly 46. In use, this combined portion of the assembly will set the proximal position of platinum coil 34 so that it can be aligned with the 3 cm marker band on microcatheters in a “T” formation to assist in implant positioning and detachments. The individual components (coil 34, coil 38 and ring assembly 46) can be adjusted relative to one another for tracking, flexibility and push, but preferably the overall combination is approximately 30 mm to approximately 32 mm to assist in fluoroscopic alignment with the microcatheter marker band located approximately 3 cm from the microcatheter tip.

Positioned (inserted) within ring assembly 46 is polyimide tube 54. Polyimide tube 54 can range in length from about 3 mm to about 2 cm, and preferably is about 1.2 cm in length, and with an OD of about 0.0064″ and an ID of about 0.0044 inches, but can range in size to as small as about 0.0025″ to as large as the thinnest available wall for a particular OD. By adjusting the wall thickness and OD of the polyimide tube, the flexibility of the overall tip may be adjusted from softer to stiffer. During assembly, the tube 54 is inserted into coil 38 until its distal end extends approximately 1 mm distally from the distal end of ring 46. In alternate embodiments, tube 54 can be inside coil 38 or flush with ring 46. It can range to an OD that just fits into the ring assembly or is significantly smaller at about 0.003″. The proximal region of the polyimide tube 54 is glued to coil 38 with adhesive joint 56 which can be a UV, thermal or melt bond constructed from materials such as adhesive or PET.

PET shrink tubing 58 covers the distal portion of the hypotube taper 26 and completely covers the coils 28, 34, 38 and 44. In some embodiments, the tubing 58 will have a vent to prevent rupture during ETO sterilization. Since the PET is a shrink tubing, it is sized to easily slide over the coils and onto the taper 26 of the hypotube. The PET shrink tubing is thin walled with for example an inner diameter of about 0.014″. Although PET shrink tubing can be utilized, other shrink tubes with thin walls may be used or the assembly may be covered using other plastics in combination with reflow techniques. The hypotube 22 in combination with the coil assembly (28, 34, and 38) and ring assembly 46 form the cathode (-) or ground electrode for the electrical circuit. The electrical ground contact is the exposed (non-insulated) region of the outer shaft 12 since that is part of the electrical circuit.

Extending through outer shaft 12 is the delivery member 14 which forms the inner core and is preferably constructed of a nitinol or stainless-steel wire 60, and preferably has a diameter of between about 0.001″ and about 0.004″, and preferably about 0.002″, and coated with a thin layer of polyimide 62 for electrical insulation. Other insulative materials can be utilized, such as PTFE. Delivery member 14 extends from distal end 64 to proximal end 66, with a proximal region not coated (non-insulated). The wire 60 can be nitinol or stainless steel. At the distal region 64, a section of the polyimide 62 is removed and is partially replaced with a melted PET 68 leaving an exposed section of nitinol with distal end 72. The exposed section forms an electrolytic detachment zone 70. The distal end of the inner core wire 60 is bent to form a J shape 74 that fits into the locking portion (opening) of the occlusive device (implant) as shown in FIGS. 7A and 7B and described below.

The inner core delivery member 14 has a centering member, e.g., centering tube or coil, 76 at a distal end that slides over (positioned over) the wire 14 and centers it in polyimide tube 54. The centering member 76 is a small coil made of stainless steel or a short polyimide tube and is sized to slide over delivery member 14 inside ring assembly 46 and has a length of between about 0.002″ and about 0.045″, and preferably less than about 0.020″. The centering member 76 and delivery member 14 are connected to the ring assembly 46, with PET melt joint 78. The distal end 80 of the PET joint 78 forms the proximal end of the detachment zone 70 as shown in FIG. 2A. The proximal end of the detachment zone 70 can also be denoted by the distal end of polyimide coating 62. Although PET is used for joint 78, other materials such as adhesive or epoxy can be used. The distal end of the detachment zone 70 is formed by the proximal end of the melted PET 68 which is over the J-tip of delivery wire 60. An exposed conductive wire portion is thus between the non-conductive PET 78 and 68.

The delivery member 14 extends through polyimide insulating tube 54, extending the entire length of the outer shaft 12 and proximally thereof, e.g., extending a length of approximately 1.2 cm proximal to outer shaft 12. At the proximal end of the inner delivery member 14, another section of polyimide 62 is removed for approximately 3 mm to expose a conductive nitinol or bare wire section 82 of delivery wire 60 terminating at proximal end 66. Thus, a proximal portion of the delivery wire 60 is not covered by the polyimide coating 62. The polyimide coating 62 extending over the delivery core wire 60 insulates the core wire 60. A distal portion of the polyimide coating 62 is removed at the detachment zone 70 at the distal region as described above. Thus, delivery wire 60 has an exposed (non-insulated) distal region for detachment and an exposed (non-insulated) proximal region for electric connection as described below.

Parallel or substantially parallel to the section of delivery wire 14 that extends proximal of the outer shaft 12 is nitinol wire 84. Nitinol wire 84 is preferably about 0.004″ in diameter and in the range of about 18 mm to about 22 mm in length with a preferred length around 20 mm. Wire 84 is inserted (positioned) into the proximal end of the hypotube 22 of outer shaft 12 and approximately 9 mm of wire 84 extends proximally out of hypotube 22. Wire 84 serves as a flexible member although it provides stiffness for recovery to allow the proximal end of the delivery device to bend and recover/straighten in flexible region 98 (FIG. 2B) without kinking/breaking. In some embodiments, it can bend to 90 degrees at a bend radius of about 0.005″ and fully recover to straight.

Covering the proximal end of wire 84 is polyimide tube 86. Polyimide tube 86 can have an inner diameter of about 0.0045″, an outer diameter of about 0.0055″ and a length of between about 8 mm to about 12 mm, and preferably about 10 mm. Adhesive joint 88 is provided at the proximal end of polyimide tube 86 so that wire 84 cannot exit tube 86. Polyimide tube 86 is used to isolate wire 84 from other metal parts e.g., delivery wire 14 and contact 92, but a coated/insulated nitinol wire with covered ends may also be used instead of polyimide tube 86. An adhesive or melt joint 90 is provided at the proximal end of hypotube 22 to adhere the polyimide tube 86 and delivery wire 14 to hypotube 22. Alternatively, wire 84 can be left exposed and a polyimide tube may be placed over delivery wire 14 so that it extends into hypotube 12 so as to provide additional coating protection to delivery wire 14 during bending. Such a tube can also be used in embodiments where wire 84 is not left exposed.

Parallel or substantially parallel to a proximal region of core wire 14 and radially spaced therefrom is electrical contact 92. Conductive member (contact) 92 extends along the longitudinal axis and is proximal of wire 84 and proximal to the proximal end 20 of hypotube 22. Electrical contact 92 is in the form of a nitinol wire having rounded ends and with a diameter of between about 0.004″ and about 0.012″, and preferably about 0.008″, and a length of between about 8 mm and about 14 mm″, and preferably about 11 mm with preferably rounded ends. If the ends are not rounded, adhesive or PET may be added to round the ends. Although nitinol is the preferred material for the electrical contact, it is understood that the contact may be constructed from any electrically conductive material and not limited to nitinol.

Electrical contact 92 is configured to interface with a corresponding electrical contact in the power supply (not shown). Wire 92 is in direct contact with exposed proximal section 82 of delivery (core) wire 60 to form an electrical contact that is configured to interface with a corresponding electrical contact in the power supply. Wire 92 is in electrical contact with the exposed proximal region of delivery wire 14 adjacent proximal end 66 and along a length. Wire 92, when coupled to delivery wire 60 of delivery member 14, will comprise the anode (+) of the electrolytic circuit when the delivery wire 14 is operatively coupled to the power supply to apply energy for electrolytic detachment. As noted above, wire 92 is not in electrical contact with wire 84 due to the insulated polyimide tube 86 and joint 88.

Note in an alternate embodiment, a connection between exposed section 82 of delivery wire 14 and conductive member 92 is made by providing element 92 as a hypotube and the end of section 82 of the delivery wire is bent into a U and positioned inside an inner lumen of the hypotube 92 to make electrical contact and reduce the outer diameter or profile of the proximal assembly.

Covering (overlying) polyimide tube 86, exposed section 82 of delivery wire 14 and a portion of electrical contact (nitinol wire) 92 is shrink tubing 94. The shrink tubing 94 can be made of PET or other material and can have an inner diameter of between about 0.010″ and about 0.016″, and preferably about 0.014,″ and a length between about 10 mm and about 25 mm, and preferably about 15 mm. The tube 94 is positioned so that about 2 mm extends beyond the proximal end 66 of core wire 60 of delivery member 14, leaving approximately 5 mm of electrical contact (nitinol wire 92) exposed proximal to the proximal end of shrink tubing 94, once it is shrunk. The distal end of the shrink tubing 94 can continue over the proximal end of hypotube 22 or it can end at the proximal end of hypotube 22 or can end proximal of the proximal end of hypotube 22 as shown in FIG. 1 to make the overall OD of the joint smaller. Covering a portion of PET shrink tubing 94 is a section of Cobalt Polymer Palladium Pebax shrink tubing 96. Shrink tubing 96 extends over shrink tubing 94 and in the illustrated embodiment, terminates at a proximal end distal of the proximal end of tubing 94 and extends distally over a proximal end of hypotube 22 to terminate distally at a region distal of the proximal end of hypotube 22. Shrink tubing 96 can also have a region 96a contacting delivery member 14 as shown in FIG. 2B. Shrink tubing 96 can have an inner diameter of between about 0.010″ and about 0.016″, and more preferably about 0.014″, and a length of between about 10 mm and about 25 mm, and more preferably about 20 mm before shrinking. Other materials for shrink tubing 94 and 96 are also contemplated. Shrink tubing 94, when shrunk, couples electrical contact 92 and exposed section 82 of delivery member 14 so they are in electrical contact. Although two shrink tubes are used in the design, the delivery wire can be built using a single shrink tubing or coating in order to reduce the outer diameter of the region covering the proximal end of hypotube 22. FIG. 2B shows an enlarged cross-sectional view of the proximal end of the delivery device 10 having proximal flexible section 98. The proximal flexible section 98 is defined as the region between distal end 100 of electrical contact wire 92 and proximal end 20 of hypotube 22. This flexible section 98 is formed by the configuration and components of the delivery device 10 at the proximal region as the delivery member 14 extends proximally of the hypotube 22 and the electrical contact 92 extends proximally of the delivery member 14 and adjacent and in abutment thereto, and is axially spaced from the hypotube 22, with attachment of contact 92 achieved through shrink tubing 94 and 96. The hollow aspect of the hypotube rather than a solid component also enhances flexibility. Electrical contact 92 is of sufficient size for contact with the controller. The spacing of contact 92 from the proximal end of hypotube 22 enables provision of a flexible region as there are more flexible components between contact 92 and hypotube 22 to thereby form the flexible region 98. As noted above, wire 84, extending through flexible region 98, provides sufficient stiffness for recovery/straightening while still having sufficient flexibility so as not to restrict or impede flexing of flexible region 98. The flexible region (section) 98 allows electrical contact wire 92 to move from neutral (straight) position 104 to a lateral (bent or flexed) position 106a or 106b with respect to the longitudinal axis. The advantage of this flexible section 98 is described below in conjunction with insertion through the delivery sheath. Note these flexible sections in preferred embodiments have high flexibility and short lengths and thus provide limited pushability. They provide electrical contact and an atraumatic proximal end that allows easy insertion through the sheath bend.

The delivery wire 14 has a J-tip at a distal end to engage the proximal tube 122 of the implant 120 as shown in FIGS. 7A and 7B. The implant 120 has a tube 122 at a proximal end (as described above and in the '611 and '012 patents) having an opening 124 (or exposed inner lumen of tube 122) in which the J-tip 74 is positioned to mechanically couple the delivery wire 14 and implant 120. The features and length of opening 124 corresponds to the shape of the J-tip 74. The J-tip 74 remains with the implant 120 in the patient's body when the implant is electrically detached at the detachment zone from the delivery wire 14.

FIGS. 7A and 7B show the above-described coaxial, bi-polar occlusive implant delivery device 10 with its flexible proximal section 98 and J-tip 74 connected to implant 120. To form the locking connection, a coil 126 fixed distal to the detachment zone 70 and proximal to the J shape section of the delivery wire 60 is fitted into opening 124 in the nitinol tube to form a lock and an epoxy is applied to form a solid joint. PET 68 electrically isolates the delivery wire J-tip from coil 126. A portion of the melted PET 68 is left exposed so that the proximal end will form the distal end 72 of detachment zone 70. The proximal end of the detachment zone is at PET joint 80.

The distal tip of delivery wire 60 can in alternate embodiments be straight with a ball tip or a bulbous tip formed of melted PET (not shown) or a coil instead of the J shape. Like the J shape, variations on the design would be configured to snap or press fit into the nitinol lock tube 122 of implant 120 followed by an epoxy joint for permanent fixation.

FIG. 8 shows an alternate embodiment for connection of the delivery device to the implant. The delivery wire 14′ of delivery device 10′ is configured with a straight tip rather than the J-tip design and is locked directly to implant 120 without use of nitinol lock tube 122. The straight tip can include a ball end. As shown, in this embodiment, implant 120 has a platinum/tungsten coil 125 running through its interior. At the proximal end, coil 125 has gapped section 127 and at least one proximal loop of wire 135 bent or wound to a smaller diameter so as to prevent the distal tip of delivery wire (ball tip or coil) from slipping out of the implant 120.

The delivery wire 14′ of delivery device 10′ has a straight tip 128 covered in melted PET 68 having a proximal end 131 and a distal end 130. As in the above design of FIG. 7A, proximal end 131 of the PET section acts as the distal end of the detachment zone 70. Centering coil 132 which can be made of platinum/tungsten wire for example, as well as other materials, is about 0.001″ to about 0.002″ in diameter wound into a coil shape about 0.003″ to about 0.0075″ long or longer is positioned over and attached to PET 68 of straight tip 128 with joint 134. Joint 134 can be made using melted PET or adhesives such as epoxy. Centering coil 132 will serve to center the delivery wire 14′ relative to implant 120 as well as to provide a bonding interface between the straight tip 128 and implant 120. In other embodiments, centering coil 132 and joint 134 can be left off of straight tip 128 and need not be provided. Delivery wire 14′ is inserted directly into implant 120 so that the centering coil 132 is sitting within coil 125 of implant 120 just distal of the most proximal smaller loop. Epoxy 136 is then applied to lock the delivery wire 14′ to implant 120. The epoxy will fill gapped section 127 and absorb into a portion of the implant 120 wall to form a solid bond. Note coil 132 is electrically isolated from delivery wire metal tip 128 and coil 125. Note in some embodiments coil 132 does not need to be electrically isolated from the implant as long as it is isolated from tip 128.

Although the implant 120 in FIG. 8 lacks nitinol lock tube 122 as in the embodiment of FIG. 7A, the coil concept described in FIG. 8 additionally or alternatively can be configured to press or snap fit into a lock tube 122 followed by an epoxy 136 joint for strength. Also, for a ball-end wire joint, either metal or plastic (PET) can be used instead of coil 132 to create the connection between the delivery wire 14′ and implant 120.

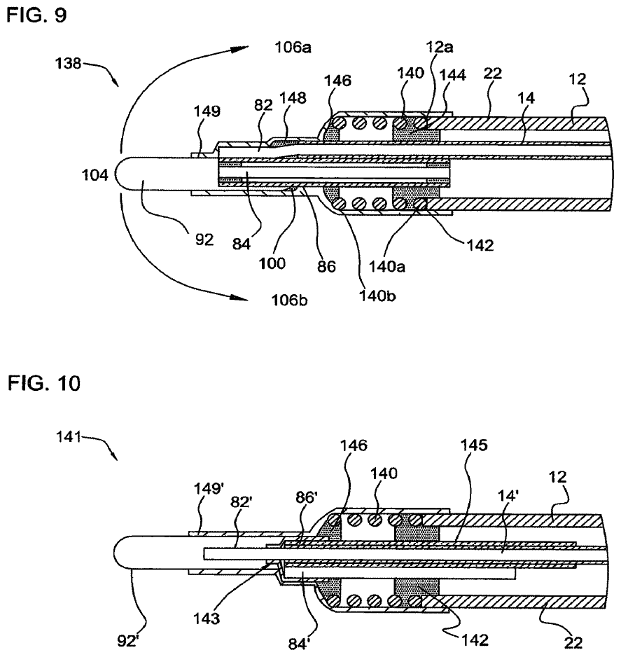

FIG. 9 illustrates another embodiment of the flexible proximal section of the bi-polar delivery system generally referred to by reference number 138. Attached to the proximal end of outer shaft 12, i.e., to the proximal end of hypotube 22, is coil 140 which serves as the flexible region. Coil 140 can have an inner diameter and outer diameter similar to hypotube 22 with a length that can range from about 2 mm up to about 8 mm, and preferably about 4.5 mm. The coil 140 can be open or closed pitch or transition between open and closed pitch. The coil 140 can be made out of stainless steel, nitinol, plastic or other suitable materials. Joint 142 attaches coil 140 to hypotube 22. Joint 142 can be created with a weld, adhesive, epoxy or PET material. Distalmost winding 140a of coil 140 can in some embodiments abut the proximalmost end 12a of hypotube 22 and can be attached by joint 142, e.g. welded thereto.

Extending through coil 140 is inner core wire 14, nitinol parallel wire 84 and polyimide tube 86 positioned over wire 84. As in the embodiment of FIG. 2B, wire 84 does not restrict the flexing or impede flexing of the flexible proximal portion. Wire 84 changes the flexibility of the region. It works in conjunction with coil 140. Wire 84 aids in flexing and recoil, enabling the assembly to return to the straight condition. In this embodiment, wire 84 is completely inside of polyimide tube 86 (in FIG. 2B it extends distally of polyimide tube 86) and the wire 84 and tube 86 are partially inserted into outer shaft 12 (hypotube 22). An additional short section of polyimide tube (not shown) can be placed around coated nitinol wire 14 where it enters the proximal opening of hypotube 22 so as to protect the outer polyimide coating of wire 14 from scraping which may remove the outer layer of polyimide thus exposing the nitinol wire directly to the outer shaft 12 (hypotube 22) and creating metal to metal contact. Both delivery member 14 and tube 86/wire 84 can be attached to the outer shaft 12 and coil 140 by adhesive or melt joint 144. The delivery member 14 and tube 86/wire 84 can also be attached proximal to coil 140 by another adhesive or melt joint 146. Note polyimide tube 86 can alternatively terminate at the proximal end of hypotube 22 while wire 84 continues into hypotube 22 (as in FIG. 2B) or it can be left off completely.

In this embodiment, both the inner core delivery wire 14 of the delivery device and nitinol wire 84/polyimide tube 86 preferably have approximately the same length (distance) extending proximally from the outer shaft 12 so they terminate at the same transverse axis, i.e., same transverse plane, however, the lengths/distances can vary and do not need to be equal. In this illustrated configuration, inner core wire 14 and nitinol wire 84/polyimide tube 86 extend approximately about 7 mm proximal of the proximal end (proximalmost coil 140b) of coil 140. The coil 140 provides for more flexibility than the more rigid hypotube. As in the embodiment of FIG. 2B, the contact 92 terminates at a distal end 100 proximal of the hypotube 22 so it is axially spaced therefrom. Wire 84 provides flexibility as described above with regard to the embodiment of FIG. 2B.

Electrical contact 92 is positioned so that the proximal end of the contact is about 1 mm to about 2 mm away from the proximal end of coil 140. In this configuration, the exposed section of electrical contact 92 is approximately 3.5 mm in length although it can be longer or shorter if needed. The electrical contact 92 is in direct contact with exposed section of 82 of inner core delivery wire 14. The electrical contact in some embodiments can overlap about 7 mm with wire 84, although other dimensions are also contemplated. An adhesive or melt joint 148 can be used to tack the electrical contact 92, inner core delivery wire 14, and nitinol wire 84/polyimide tube 86 together. Covering the components is shrink tubing 149 which may be made of Pebax (Cobalt Polymer), PET, or other suitable shrink tubing material. Tubing 149 extends over a proximal end of hypotube 22, coil 140, joint 146, joint 148, a proximal exposed section 82 of wire 14 and a distal section of electrical contact 92.

FIG. 10 illustrates another embodiment of the flexible proximal section of the bi-polar delivery system generally referred to by reference number 141. In this embodiment, tube 86′ (which is the same composition and function as tube 86 of FIG. 9) covers inner core delivery wire 14′ (which has the same function as wire 14 of FIG. 9). Tube 86′ extends from the distal end of bare (exposed) wire section 82′ of wire 14 into shaft 12 (hypotube 22) for a length, terminating distally inside hypotube 22 at between approximately 1 mm to approximately 10 mm. This use of tube 86′ in this embodiment provides added protection against abrasion to inner core delivery wire 14′. In this embodiment, the dimensions of tube 86′ can be decreased to about 0.0025″/0.0031″ and nitinol wire 84′ down to about 0.003″. Since wire 84′ is bare in this embodiment, i.e., is not covered by an insulating tube as in the embodiment of FIG. 9, shrink tubing 143, covering the distal end of wire 84′, is used to insulate wire 84′ from electrical contact 92′. Wire 84′ aids in flexing and recoil in the same manner as wire 84 of FIG. 9. Electrical contact 92′ functions in the same manner as electrical contact 92 of FIG. 9 as contact 92′ and delivery wire 14′ form the anode and the hypotube 22 of outer shaft 12 forms the cathode. The shrink tubing 143 as shown extends over a portion of wire 84′ and insulative polyimide tube 145 which is placed around insulative polyimide tube 86′ surrounding delivery wire 14. Tubings 145 and 86′ terminate at a proximal end within shrink tubing 143.

Joint 142 attaches coil 140 at a distal end to hypotube 12 and delivery wire 14′. Joint 146 attaches coil 140 at a proximal end to delivery wire 14′/tube 86′ and wire 84′. Shrink tubing 149, like shrink tubing 149, extends over the coil 140 and proximal of hypotube 22. Coil 140 in the illustrated embodiment has an outer diameter substantially the same as the outer diameter of the hypotube 22, although other dimensions are also contemplated.

Note in the embodiment of FIG. 10, the delivery wire is positioned closer to the central longitudinal axis of the hypotube 22, and can in some embodiments be positioned along the center line axis. The wire 14 is more radially offset from the central longitudinal axis in the embodiment of FIG. 9. In both embodiments, the nitinol wire 84 is radially offset from the central longitudinal axis. Note it is also envisioned that the delivery wire 14 and/or the nitinol wire 84 can be axially and/or radially positioned in regions within, or respect to, the hyptotube 22 (shaft 12) other than those shown in FIGS. 9 and 10. In FIG. 9, delivery wire 14 has a slight bend at the proximal end; in FIG. 10, the proximal end remains straight.

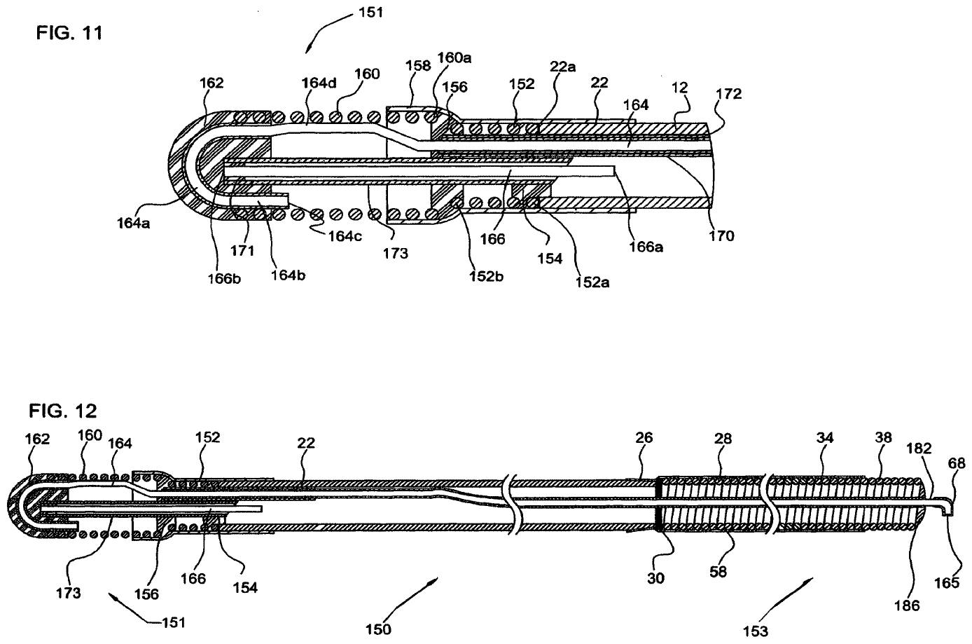

FIGS. 11 and 12 illustrate another embodiment of the delivery system. FIG. 11 illustrates the flexible proximal section of the bi-polar delivery system generally referred to by reference number 151 which forms the proximal end of delivery system 150. FIG. 12 illustrates section 151 in conjunction with the distal section of delivery system generally referred to by reference numeral 153 which forms the electrolytic detachment end. As with FIGS. 9 and 10, only the proximal section is shown in FIG. 11. The distal section can be the same as in FIGS. 1 and 2A or alternatively have the components/configuration as shown in FIG. 12. It should also be appreciated that the various embodiments of the proximal sections can be used with the distal section of FIG. 2A or alternatively used with the distal section of FIG. 12 or used with alternative distal sections described herein.

Attached to the proximal end of outer shaft 12, i.e., to the proximal end of hypotube 22, is coil 152 which serves as part of the flexible region. (Coil 152 is similar in function and configuration to coil 140 of FIGS. 9 and 10). Like coil 140, coil 152 can have an inner diameter and outer diameter similar to hypotube 22 with a length that can range from about 2 mm up to about 8 mm, and preferably about 4.5 mm., although other diameters and lengths are also contemplated. The coil 152 can be open or closed pitch or transition between open and closed pitch. The coil 152 can be made out of stainless steel, nitinol, plastic or other suitable materials. Joint 154 (like joint 142 of FIGS. 9 and 10) attaches coil 152 to hypotube 22 and to delivery wire 164 via outer tubing 170. Joint 154 can be created with a weld, adhesive, epoxy or PET material, or a combination of these methods. Distalmost winding 152a of coil 152 can in some embodiments abut the proximalmost end 22a of hypotube 22.

Extending through coil 152 is inner core wire 164 and nitinol wire 166. Surrounding core wire 164 is inner polyimide coating 172 and outer polyimide tubing 170 which is positioned over inner polyimide coating 172. Components 170 and 172 also extend through coil 152. As in the embodiment of FIG. 2B, wire 166 stiffens the assembly and has impact on overall flexibility. It improves recoil/recovery. Polyimide tube 173 surrounds wire 166 to electrically isolate wire 166 and in this embodiment, a portion of wire 166 extends distally of polyimide coating 172 (as in wire 84 of FIG. 2B extending distally of polyimide tube 86). A distal portion of the wire 166 and tube 173 extend into outer shaft 12 (hypotube 22) so distal edge 166a of wire 166 is within hypotube 22. Adhesive joint 171 is provided at the proximal end of polyimide tube 173 and the proximal end 166b of wire 166 to attach the two components at a proximal end and ensure that wire 166 cannot exit tube 173. Both delivery wire 164 and tube 173/wire 166 can be attached to the outer shaft 12 (hypotube 22) and coil 152 by adhesive or melt joint 154. The delivery wire 164 and tube 173/wire 166 can also be attached to a proximal region of coil 140 by another adhesive or melt joint 156 which is proximal of joint 154. Thus, as shown, a proximalmost winding 152b of coil 152 lies within joint 156 (although more than one winding 152b of coil 152 can lie within joint 156); a distalmost winding 152a of coil 152 lies within joint 154 (although more than one winding 152a of coil 152 can lie within joint 154). Outer and inner components 170, 172 also lie within joint 156 at their proximal ends.

The coil 152 provides for more flexibility than the more rigid hypotube. The contact for the conductive path (anode) is proximal of the hypotube 22 so it is axially spaced therefrom. This contact is in the form a coil described in detail below. Wire 166 provides flexibility and recoil/recovery in the same manner as wire 84 of the embodiment of FIG. 2B described above. Note the added material makes the assembly stiffer.

To further increase flexibility at the proximal region 151, a second proximal coil 160 is positioned proximal of coil 152 (referred to herein as the “distal coil”). The coil 160 is referred to herein as the “proximal coil.” Joint 156 can also encompass a distalmost winding 160a (or more than one winding) of coil 160 for attachment. Coil 160 is electrically isolated from coil 152 via joint 156. Shrink wrap tubing 158 also helps electrically isolate coil 160 from coil 162 as it extends over a proximal portion of hypotube 12, over coil 152, over joint 156 and over distalmost coils of coil 160. Proximal coil 160 in the illustrated embodiment has a larger outer diameter than distal coil 152 and hypotube 22. Provision of coil 160 instead of nitinol wire contact as in the embodiment of FIG. 1 makes this section more flexible, and durable and could make it easier to make contact inside the detachment controller.

Coil 160 is in electrical (conductive) contact with core wire 164 along an exposed (non-insulated) portion 164d. Core wire 164 as shown extends proximal of hypotube 12 (i.e., proximally of proximalmost edge 22a of hypotube 22), angles to contact the coil 160 and forms a loop at region 164a to extend back toward the distal end 164b to terminate at distal end 164c proximal of the hypotube 22 (and proximal of distal coil 152). Thus, the proximal end of wire 164 forms a J-shape or U-shape to provide electrical contact along one side of coil 160 as it contacts an inner surface thereof. The core wire 164, like core wire 14 and 14′ discussed above, is thin and flexible and thus provides electrical connection and detachment at the electrolytic joint while the stiffer hypotube 22 provides the pushability to push/advance the implant during delivery prior to electrolytic detachment.

Note the looped core wire 164 can be used with the various embodiments of the delivery system disclosed herein.

As discussed above, a polyimide coating 172 covers delivery wire 164 and an outer polyimide tube 170 is placed over polyimide coating 172. Together, these components 170, 172 provide for electrical insulation of delivery core wire 164 from distal coil 152. The proximal ends of components 170, 172 terminate within joint 156 and distal of the distalmost coil 160a of distal coil 160. The absence of components 170, 172 proximal of joint 156 would provide a more flexible region of the delivery wire 164 as the outer diameter is reduced.

A nonconductive material/joint 162 (adhesive or melt joint) forms a proximal end cap of the delivery system. Joint 162 attaches proximal coil 160, delivery wire 164, tubing 173 and nitinol wire 166 at proximal ends as shown.

Turning to the distal section 153 of delivery system 150 of FIG. 12, the outer shaft 12 includes a hypotube 22 and coils 28, 34, 38. These coils are the same as in FIG. 1 described above, however, the wound platinum coil 44 is not provided in this embodiment, although in alternate embodiments it could be provided. This embodiment also differs from that of FIG. 1 in that the PET shrink tubing 58 does not cover the distal coil 38, or covers only a proximal region of distal coil 38. In this manner, the coil 38, or at least a portion thereof, is exposed so that coil 38 can function as the return electrode, in electrical contact/communication with the hypotube 22 via coils 28 and 34. Thus, in this embodiment, instead of a ring assembly such as ring assembly 46 described above in connection with the embodiment of FIG. 1, the coil 38 can provide the return/cathode function. At the distal region, the hypotube 22 can have a taper 26 in a distal direction.

The above discussion of the hypotube 22 and coils 28 and 34 is fully applicable to the embodiment of FIG. 12 so for brevity further discussion of the hypotube 22, coil attachment, function, materials, and alternatives thereof is not repeated herein.

The exposed portion of coil 38 (non-insulated by shrink tube 58) can have a length of about 0.1 mm to about 10 mm. PET shrink tubing 58 covers the distal portion of the hypotube taper 26 and completely covers the coils 28 and 34. The hypotube 22 in combination with the coil assembly (28, 34, and 38) form the cathode (-) or ground electrode for the electrical circuit. The electrical contact is the exposed (non-insulated) region of the outer shaft 12 since that is part of the electrical circuit.

PET melt joint 186 is provided at distal region of the wire 164 and joins the wire 164 and distal coil 38, but electrically isolates these two components. Delivery wire 164 extends distally out of the inner insulating coating 172 and distally past melt joint 186 so a delivery wire portion 182 is exposed just proximal of the J-tip 165. Stated another way, at the distal region of delivery wire 164, a section of the polyimide 172 is removed and is partially replaced with a melted PET 68 leaving an exposed section 182 of wire 164. The exposed section forms an electrolytic detachment zone like zone 70 described above. The distal end of the inner core wire 164 is bent to form a J shape 165 that fits into the locking portion (opening) of the occlusive device (implant) in the same way described above and shown in FIGS. 7A and 7B. Thus, further description of the J-tip and implant is not provided for brevity since the J-tip 165 functions in the same manner as J-tip 74. Shapes/configurations other than the J-tip, e.g., ball tip, are also contemplated as described above.

The use of the bipolar delivery system of the present invention will now be described. In use, an electrical path is created from the box/power source (not shown), through the nitinol wire 92 (or 92′ and/or coil 160) and the delivery wire 14 (or 14′ or 164) to the detachment zone 70 (or 182) at a distal end thereof. The electrical energy severs the joint to detach the implant from the delivery wire. The J-tip 74 (or 165) of the delivery wire (or alternate tips as disclosed herein), distal of the detachment zone 70, remains with the implant in the patient's body. The return is provided through the ring assembly 46 (or through coil 38 in the FIG. 12 embodiment) and proximally along the length of the outer shaft 12 (coils and hypotube 22). The box is preset to a maximum time limit so energy is terminated after a set time period, such as 25 seconds, although other time periods are also contemplated.

It should be appreciated that the J-tip wire can be used in monopolar electrolytic systems as well. In such systems, the J-tip would engage the tube of the implant in the same manner as J-tip 74 of FIG. 7A, and upon application of energy, the wire would detach at a region just proximal of the J-tip 74, and the J-tip 74 would detach along with the implant.

In use of the bipolar system, in the various embodiments disclosed herein, the core delivery wire 14 (or wire 14′, 164, etc.) forms the first conductive path between electrical contact 92 (or contact 92′ or coil 160 in the FIG. 12 embodiment) and electrolytic detachment zone 70. The first conductive path will form the anode (+) of the electrical circuit. A second conductive path, the cathode (−) or ground electrode for the electrical circuit, is formed from the hypotube 22 in combination with the coil assembly (28, 34, and 38) and ring assembly 46 or coil 38 of FIG. 12 as the electrical path is created from ring 48 through coils 44, 38, 34 and 28 and through hypotube 22 or created from distal coil 38 through coils 34 and 28 and through hypotube 22. The ground contact for the cathode is the exposed length of hypotube 22 (i.e., non-insulated region between tubing 96 (or tubing 149, 149′, 152) and tubing 58) which is configured to interface with a corresponding electrical contact (not shown) in the power supply (not shown) when the proximal end of the delivery wire 10 is inserted into the power supply (not shown). As described above, the first and second conductive paths are electrically isolated from one another. Blood provides an electrolytic medium e.g. conductivity between the two paths during detachment. When electrical current from the power supply (not shown) is applied, the detachment zone 70 between the delivery wire and the implant dissolves. It should be appreciated that the embodiments of FIGS. 9-12 function in a similar fashion as FIG. 1 with either the ring assembly forming the return with the hypotube 22 as in FIG. 1 or the coil 38 forming the return with the hypotube 22 as shown in FIG. 12 and the delivery wire 14, 14′, 164 forming the first conductive path from the respective contact 92, 92′, coil 160 to provide electrical energy to the electrolytic detachment joint at the distal end of the delivery wire wherein it is detached at the electrolytic joint (distal of the insulative melt joint 78, 186) leaving the J-tip 74, 165 (or other shaped tip) of the delivery wire 14, 14′, 164 with the implant.

The delivery systems disclosed herein are for use for delivering devices for treating intracranial aneurysms, however it is also contemplated that the delivery systems can be used to deliver devices through and in other body lumens in a patient.

The delivery wire of the present invention can be used with the packaging disclosed in the '611 patent and patent publication no. 20230233279. The delivery wire would engage the vascular implant tube within the bulb packaging.

Delivery sheaths for neurovascular embolization coils typically have a proximal feature such as a crimped section to prevent the coil delivery wire from moving relative to the delivery sheath during transportation. Another method for limiting coil movement is a bend in the proximal section of the delivery sheath. The delivery sheath is used to secure (lock) the delivery device for introduction of the delivery device with attached implant into the microcatheter for delivery to the patient body. The delivery device is moved proximally into the sheath as described in the '611 patent and patent publication no. 20230233279 and moved proximally within the sheath so its proximal end is exposed as for example in FIG. 5. Once exposed, the delivery sheath is placed in abutment with the proximal end of a microcatheter and the implant is advanced by the delivery device 10 (or 10′) through the microcatheter and into the patient's body for subsequent detachment of the implant.

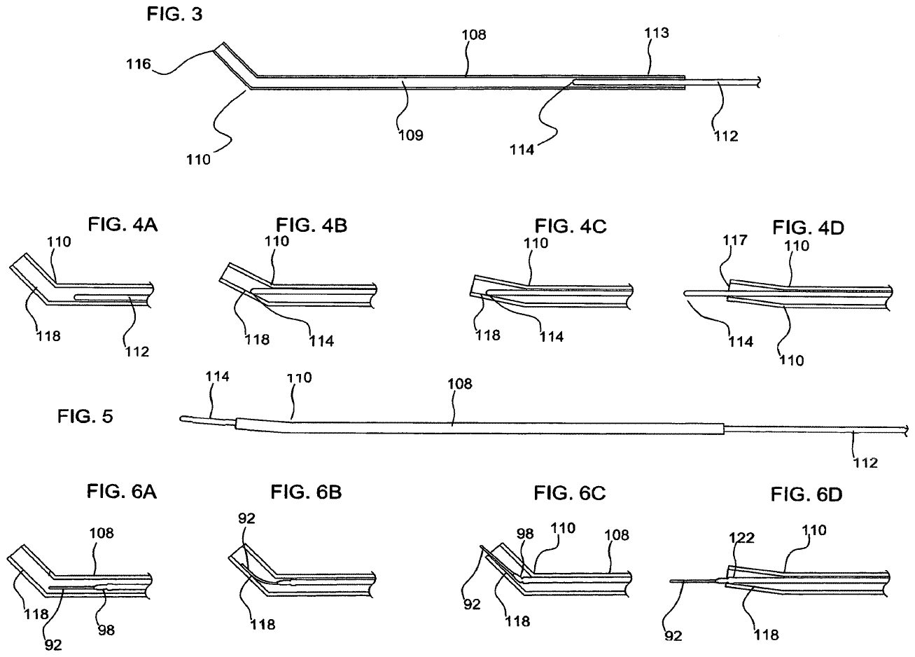

FIG. 3 shows a conventional delivery sheath 108 with a bend 110 having a delivery wire 112 (which does not have the flexible region of the present invention) introduced into its lumen 109 from the distal end 113. Delivery wire 112 is shown partially positioned therein. The delivery wire 112 has a proximal end 114 and extends through the lumen 109 of sheath 108. The delivery wire 112 is introduced into the lumen 109 from the distal end 113 of the sheath 108. The delivery sheath 108 can be made of any polymer typically used in sheath construction such as a polyimide sheath with a PTFE liner for lubricity. The sheath is typically about 100 cm long although greater or smaller lengths are also contemplated. The inner diameter of the sheath 108 typically matches the inner diameter of the microcatheter to which it is to transfer the implant, although it can be larger or smaller, if needed. The bend 110 has a curvature/radius which can be between about 3 mm and about 8 mm from the proximal end 116 of sheath 108 or at any other location along the sheath. The delivery wire 112 is constructed using either a hypotube construction or tapered core wire construction that are typical for microcoil delivery systems. In either design, the proximal end 114 is more rigid than the distal end and thus lacks the flexible region of the present invention. After passing through sheath bend 110 and forcing it to straighten, the delivery wire 112 is frictionally interlocked with the delivery sheath.

FIGS. 4A-4D show proximal end 114 of delivery wire 112 progressing through delivery sheath 108. As the proximal end 114 contacts the bend region 108 of the sheath, it will straighten the region as well as scrape against the entire length of inner wall 118 removing PTFE liner as it moves towards the proximal opening. The flaking PTFE liner will potentially cause particulate in the sterile field. FIG. 5 shows the delivery wire 112 with the proximal end 114 that has passed bend 110 and exited the proximal end of the delivery sheath 108 through proximal opening 117.

FIGS. 6A-6D show the coaxial, bi-polar wire of the present invention having a flexible proximal end region 98 progressing through delivery sheath 108. As the electrical contact 92 (or 92′ or end region 151 in the embodiment of FIG. 12) of the delivery wire progresses through the bent region of sheath 108, the electrical contact 92 will make contact with wall 118 (FIG. 6B) and flexible section 98 of delivery device 10 will bend and slide up wall 118 of delivery sheath 108 (FIG. 6C). The bend 110 will start to straighten as the flexible section 98 meets wall 118 and advances past it (FIG. 6D) with the delivery wire proximal end transitioning from flexible to more rigid at the proximal end of the hypotube 122. Because the proximal end of the hypotube 122 is covered in shrink tubing and leads with a flexible-to-rigid transition section, it will slide smoothly relative to wall 118 and gradually transition from flexible to rigid minimizing stresses on the PTFE liner or inner lumen of the sheath. The flexible regions at regions 138, 141 and 151 of FIGS. 9-12 will function in this same manner as flexible section 98.

The present invention provides electrolytic delivery systems that have sufficient stiffness to provide pushability of the implant while having sufficient flexibility to navigate the tortuous anatomy. The delivery systems of the present invention also have more flexible proximal ends to improve packaging and provide more atraumatic delivery. Note the various dimensions and range of dimensions of the components discussed herein e.g., length, OD, ID, thickness, etc. are provided by way of example as other dimensions and ranges are also contemplated to achieve the structure and function disclosed herein. Also note that materials discussed above for the various components are provided by way of example as other materials can also be utilized.

Note the J-tip and various other tips described herein can also be used for attachment of the delivery wire to the implant for mechanical decoupling instead of electrolytic detachment.

While the above description contains many specifics, those specifics should not be construed as limitations on the scope of the disclosure, but merely as exemplifications of preferred embodiments thereof. Those skilled in the art will envision many other possible variations that are within the scope and spirit of the disclosure as defined by the claims appended hereto.

Although the systems, devices, apparatus and methods of the subject invention have been described with respect to preferred embodiments, those skilled in the art will readily appreciate that changes and modifications may be made thereto without departing from the spirit and scope of the present invention as defined by the appended claims.

Elements and features shown or described in connection with one embodiment may be combined with those of another embodiment without departing from the scope of the present invention.

Where a range of values is provided, it is understood that each intervening value between the upper and lower limit of that range and any other stated or intervening value in that stated range is encompassed by the present disclosure.

It should be noted that as used herein and in the appended claims, the singular forms “a”, “and”, and “the” include plural references unless the context clearly dictates otherwise.

Throughout the present disclosure, terms such as “approximately,” “generally,” “substantially,” “around, “about” and the like should be understood to allow for variations in any numerical range or concept with which they are associated. For example, it is intended that the use of terms such as “approximately”, “generally” “substantially”, “around” and “about” should be understood to encompass variations on the order of 25% (e.g., to allow for manufacturing tolerances and/or deviations in design).

Although terms such as “first,” “second,” “third,” etc., may be used herein to describe various operations, elements, components, regions, and/or sections, these operations, elements, components, regions, and/or sections should not be limited by the use of these terms in that these terms are used to distinguish one operation, element, component, region, or section from another. Thus, unless expressly stated otherwise, a first operation, element, component, region, or section could be termed a second operation, element, component, region, or section without departing from the scope of the present invention.

Each and every claim is incorporated as further disclosure into the specification and represents embodiments of the present disclosure. Also, the phrases “at least one of A, B, and C” and “A and/or B and/or C” should each be interpreted to include only A, only B, only C, or any combination of A, B, and C.

What is claimed is:

1. A system for delivering a vascular implant into a body lumen of a patient, the system comprising:
an elongated delivery device having an outer member having a lumen and a delivery member positioned within the lumen of the outer member, the delivery member having an engagement member at a distal portion and a detachment zone spaced proximally of the engagement member, the engagement member engageable with a first engagement portion of the implant, the proximal region of the delivery member extending proximally of the outer member;
wherein energy is applied to the delivery member to detach the engagement member from the delivery wire and separate the implant along with the engagement member from the delivery member.

2. The system of claim 1, wherein bipolar energy is applied to detach the delivery member from the implant wherein the delivery member forms an anode and the outer member forms a cathode.

3. The system of claim 1, wherein the engagement member has a J-tip engageable with the implant.

4. The system of claim 3, wherein the implant includes a tube at a proximal end having a recess, and the recess forms the first engagement portion dimensioned to receive the J-tip.

5. The system of claim 1, wherein the outer member includes a hypotube forming a conductive member.

6. The system of claim 5, wherein a proximal region of the delivery wire is offset from a central longitudinal axis of the conductive member.

7. The system of claim 1, further comprising a conductive member positioned radially of the delivery member, the delivery member and conductive member in electrical contact and forming an anode of a circuit when the conductive member is electrically coupled to a power supply.

8. The system of claim 1, further comprising a flexible wire at a proximal region of the delivery device, the flexible wire extending proximally of the outer member to enable bending of the proximal region without kinking.

9. The system of claim 7, further comprising a flexible wire positioned radially of the delivery member and distally of the conductive member, and the conductive member terminates proximally of the outer member.

10. The system of claim 1, further comprising a first insulator positioned proximal of the detachment zone and a second insulator positioned on the engagement member to insulate the engagement member and prevent electrical energy flow to the implant.

11. The system of claim 1, wherein a proximal region of the delivery device is more flexible than an intermediate region of the delivery device.

12. The system of claim 1, further comprising a ring assembly at a distal region of the delivery member, the delivery member extending within the ring assembly.

13. The system of claim 1, wherein the delivery member at a proximal end loops to form a U-shape.

14. The system of claim 13, further comprising a first coil at a proximal region of the delivery system, the U-shaped end of the delivery member in electrical contact with the coil.

15. The system of claim 14, further comprising a second coil distal of and electrically isolated from the first coil.

16. A system for delivering a vascular implant into a body lumen of a patient, the system comprising:
an elongated delivery member having an engagement member at a distal portion and a detachment zone spaced proximally of the engagement member, the engagement member engageable with a first engagement portion of the implant;
an outer member, the elongated delivery member positioned within the outer member and extending distally and proximally of the outer member; and
a conductive member positioned proximally of the outer member and in contact with the delivery member for connection to a power source;
wherein energy is applied to the conductive member and to the delivery member to detach the engagement member from the delivery wire and separate the implant from the delivery member.

17. The system of claim 16, wherein the system is a bipolar system and the delivery member forms the anode and the outer member forms the cathode.

18. The system of claim 16, further comprising a wire extending distally of the conductive member and a distal end of the conductive member terminates proximally of the outer member and distally of a proximal end of the delivery member.

19. The system of claim 18, wherein the wire has a first portion within the outer member and a second portion proximal of the outer member.

20. The system of claim 16, wherein the delivery member at a proximal end loops to form a U-shape.

21. The system of claim 16, wherein the conductive member is a coil and the delivery member extends through the coil.

22. A system for delivering a vascular implant into a body lumen of a patient, the system comprising:
an elongated delivery device having an outer member having a lumen and a delivery member positioned within the lumen of the outer member, the delivery member having an engagement member at a distal portion and a detachment zone spaced proximally of the engagement member, the engagement member engageable with a first engagement portion of the implant, the delivery member having a ring assembly positioned around a distal portion thereof and having a first portion within the outer member and a second portion extending distally of the outer member;
wherein energy is applied to the delivery member to detach the engagement member from the delivery wire and separate the implant along with the engagement member from the delivery member.

23. The system of claim 22, wherein the ring assembly includes a ring having an arm extending proximally within the outer member.

24. The system of claim 23, wherein the detachment zone is distal of the ring assembly.

25. The system of claim 22, wherein the delivery member at a proximal end loops to form a U-shape.
